(19) United States
(12) Patent Application Publication
(10) Pub. No.: US 2019/0099083 A1
(43) Pub. Date: Apr. 4, 2019

(54) INFORMATION PROCESSING APPARATUS AND INFORMATION PROCESSING METHOD

(71) Applicant: CANON KABUSHIKI KAISHA, Tokyo (JP)

(72) Inventor: Toshifumi Fukui, Yokohama-shi (JP)

(21) Appl. No.: 16/141,681

(22) Filed: Sep. 25, 2018

(30) Foreign Application Priority Data
Sep. 29, 2017 (JP) 2017-190719

(51) Int. Cl. A61B 5/00; G06T 7/11; G06T 7/00

(52) U.S. Cl. CPC A61B 5/0095; G06T 7/11; G06T 7/0012; A61B 5/7485; A61B 5/7282; A61B 2576/02; A61B 5/4312; A61B 5/004; G06T 2207/30096; G06T 2207/30101; G06T 2207/30068; A61B 5/489

(57) ABSTRACT

An information processing apparatus includes: an image acquisition unit that acquires first image data for a first object and second image data for a second object that corresponds to a portion that forms a pair with the first object; a setting unit that sets a region of interest for the first image data and a region of interest for the second image data; an index value acquisition unit that acquires an index value that represents a characteristic feature within the region of interest set for the first image data and an index value that represents a characteristic feature within the region of interest set for the second image data; and an acquisition unit that, based on the results of a comparison of the index values, acquires information indicating the possible presence of a lesion in at least one of the first object and the second object.

### BACKGROUND OF THE INVENTION

### Field of the Invention

The present invention relates to an apparatus that processes an image that reflects information on the interior of an object.

### Description of the Related Art

Photoacoustic imaging is known as a technique for imaging structural information and physiological information, i.e., functional information, on the interior of an object.

When an organism, i.e., an object, is irradiated with light, e.g., laser light, acoustic waves (typically ultrasound waves) are produced when the light is absorbed by biological tissue in the interior of the object. This phenomenon is referred to as a photoacoustic effect, and the acoustic waves produced by the photoacoustic effect are referred to as photoacoustic waves. Since the absorptivity for the light energy varies with each tissue constituting the object, the acoustic pressure of the generated photoacoustic waves also varies. In photoacoustic imaging, the generated photoacoustic waves are received with a probe and characteristic information on the interior of the object can then be acquired by mathematical analysis of the received signal.

In order to recognize abnormalities from the images obtained in this manner and make an accurate diagnosis, experience must be gained after receiving specialized training. On the other hand, the diagnostic imaging apparatus that outputs such an image may be provided with a mechanism that supports interpreting the information obtained from the image and recognizing an abnormal condition, e.g., a disease or lesion.

As a technique related to this, for example, Japanese Patent Application Laid-open No. 2002-158853 discloses a method of facilitating diagnosis by respectively imaging the left and right breasts using an x-ray mammography apparatus and displaying the obtained images side-by-side.

### SUMMARY OF THE INVENTION

The technique disclosed in Japanese Patent Application Laid-open No. 2002-158853 is directed to an x-ray mammography image. An X-ray mammography apparatus is a technology for visualizing substances not present in healthy breasts, such as calcified regions, thus facilitating diagnosis using human vision. However, a problem here is that it is extremely difficult to quickly discriminate the presence of a disease or lesion with human vision in a case where the blood vessels present in the breast are imaged, such as by photoacoustic imaging apparatus.

The present invention was achieved considering this problem in the prior art, and an object of the present invention is to provide an information processing apparatus that estimates the possible presence of a lesion based on image data that expresses object information.

In order to solve the aforementioned problem, the information processing apparatus according to the present invention includes: an image acquisition unit that acquires first image data for a first object and second image data for a second object that corresponds to a portion that forms a pair with the first object; a setting unit that sets a region of interest for the first image data and a region of interest for the second image data; an index value acquisition unit that acquires an index value that represents a characteristic feature within the region of interest set for the first image data and an index value that represents a characteristic feature within the region of interest set for the second image data; and an acquisition unit that, based on the results of a comparison of the index values, acquires information indicating the possible presence of a lesion in at least one of the first object and the second object.

In addition, the information processing method according to the present invention includes the steps of: acquiring first image data for a first object and second image data for a second object that corresponds to a portion that forms a pair with the first object; setting a region of interest for the first image data and a region of interest for the second image data; acquiring an index value that represents a characteristic feature within the region of interest set for the first image data and an index value that represents a characteristic feature within the region of interest set for the second image data; and acquiring, based on the results of a comparison of the index values, information indicating the possible presence of a lesion in at least one of the first object and the second object.

The present invention thus enables the estimation of the possible presence of a lesion based on image data that expresses object information.

Further features of the present invention will become apparent from the following description of exemplary embodiments with reference to the attached drawings.

### BRIEF DESCRIPTION OF THE DRAWINGS

FIG. 1 is a schematic diagram that shows the principle underlying photoacoustic imaging;

FIG. 2 is a diagram that shows the absorption spectra of oxyhemoglobin and deoxyhemoglobin;

FIG. 3 is a functional block diagram of a photoacoustic apparatus according to a first embodiment;

FIG. 4A and FIG. 4B are examples of images acquired with the photoacoustic apparatus;

FIG. 5 is an example of the comparison items used for diagnostic support using photoacoustic data;

FIG. 6 is a flow chart that shows the flow in the diagnostic support process;

FIG. 7A and FIG. 7B are schematic diagrams that show the regions of interest set in images;

FIG. 8 is an example of a screen that displays, as a left and right pair, photoacoustic data that has been imaged;

FIG. 9 is an example of metadata that holds information for an image;

FIG. 10 is an example of a table holding information relating to a reference point; and

FIG. 11 is an example of a graph that compares the number of blood vessel branch points for regions of interest.

### DESCRIPTION OF THE EMBODIMENTS

Embodiments of the present invention are described below with reference to the drawings. However, the dimensions, materials, shapes, relative positions, and so forth of the constituent components described in the following may be modified as appropriate depending on various conditions and the constitution of the apparatus used by the invention. Thus, the scope of this invention should not be construed as being limited to the description that follows.

The present invention relates to a technique that detects acoustic waves propagated from an object and that produces and acquires characteristic information about the interior of the object. Thus, the present invention may be embodied as a photoacoustic apparatus or a method for controlling same or as a method for acquiring information about an object. The present invention may also be embodied as a program that executes these methods on an information processing apparatus provided with hardware resources, e.g., a CPU, memory, and so forth, and/or as a computer-readable non-transitory storage medium that stores this program.

The present invention may also be embodied as an information processing apparatus and/or an information processing method, in each instance that performs diagnostic support based on the object image acquired by a photoacoustic apparatus and/or an object information acquisition apparatus.

The photoacoustic apparatus according to the present invention is an apparatus that receives the acoustic waves generated within an object upon the irradiation of the object with light (electromagnetic radiation) and that utilizes the photoacoustic effect to acquire characteristic information about the object as image data. In this case, the characteristic information is characteristic value information that corresponds to each of a plurality of positions within the object and that is produced using the received signal obtained by receiving the photoacoustic waves.

The characteristic information acquired by the photoacoustic measurement is a value that reflects the absorptivity for the light energy. This includes, for example, the generation source for the acoustic waves produced by the irradiation with light, the initial acoustic pressure within the object or the light energy absorption density or absorption coefficient derived from the initial acoustic pressure, and the concentration of the material constituting the tissue.

In addition, spectral information, i.e., the concentration of the material constituting the object, is obtained based on the photoacoustic waves generated using light at a plurality of different wavelengths. The spectral information may be the oxygen saturation; a value provided by weighting the oxygen saturation with the intensity of, e.g., the absorption coefficient; the total hemoglobin concentration; the oxyhemoglobin concentration; or the deoxyhemoglobin concentration. It may also be the glucose concentration, collagen concentration, melanin concentration, or the volume fraction of fat or water.

The embodiment described in the following sets out a photoacoustic imaging apparatus that performs imaging by irradiating an object with light at a wavelength at which hemoglobin is presumed to be an absorber and acquiring data on the distribution and morphology of the blood vessels within the object and data on the oxygen saturation distribution in these blood vessels.

A two-dimensional or three-dimensional characteristic information distribution is obtained based on the characteristic information at each position within the object. The distribution data can be produced as image data. The characteristic information may be obtained as distribution information for the individual positions within the object rather than as numerical data. That is, it is distribution information such as the initial acoustic pressure distribution, energy absorption density distribution, absorption coefficient distribution, or oxygen saturation distribution.

In this Specification, the acoustic waves are typically ultrasound waves and include elastic waves referred to as sound waves and photoacoustic waves. The electrical signal converted from the acoustic waves by, for example, a probe, is also referred to as an acoustic signal. However, the use of ultrasound wave or acoustic wave in the description in this Specification should not be construed as a limitation on the wavelength of these elastic waves. The acoustic waves produced by the photoacoustic effect are referred to as photoacoustic waves or optical ultrasound. The electrical signal derived from photoacoustic waves is also referred to as a photoacoustic signal. In this Specification, the photoacoustic signal is a concept that includes both analog signals and digital signals. Distribution data is also referred to as photoacoustic image data and reconstructed image data.

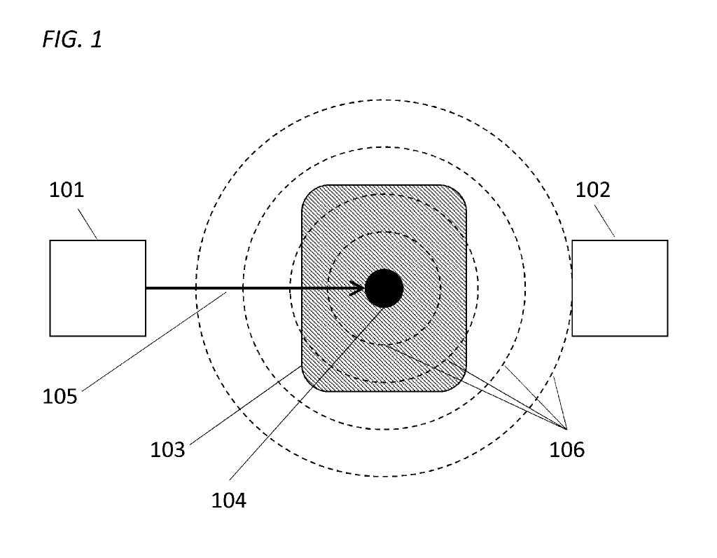

The principle of photoacoustic imaging is described in the following with reference to FIG. 1.

FIG. 1 contains a light source 101, an acoustic wave probe 102, an object 103, a photoabsorber 104 present in the interior of the object, light 105 irradiated from the light source, and acoustic waves 106 generated from the photoabsorber 104.

When light 105 is irradiated from the light source 101 onto the object 103, the light 105 reaches into the interior of the object 103 and is absorbed by the photoabsorber 104. When this occurs, acoustic waves 106 are generated and travel to the acoustic wave probe 102. The acoustic waves 106 received by the acoustic wave probe 102 are converted into electrical signals and imaged by a computer based on the timing of light irradiation, the propagation velocity within the object, and so forth, with display on a screen.

When the hemoglobin in erythrocytes is targeted as the photoabsorber 104, irradiation is carried out using light at a wavelength that is specifically absorbed by hemoglobin. The image yielded by doing this shows the uneven distribution of hemoglobin within the organism. Since hemoglobin is present in large amounts in erythrocytes, an image can then be obtained of the blood vessels wherein the erythrocytes reside.

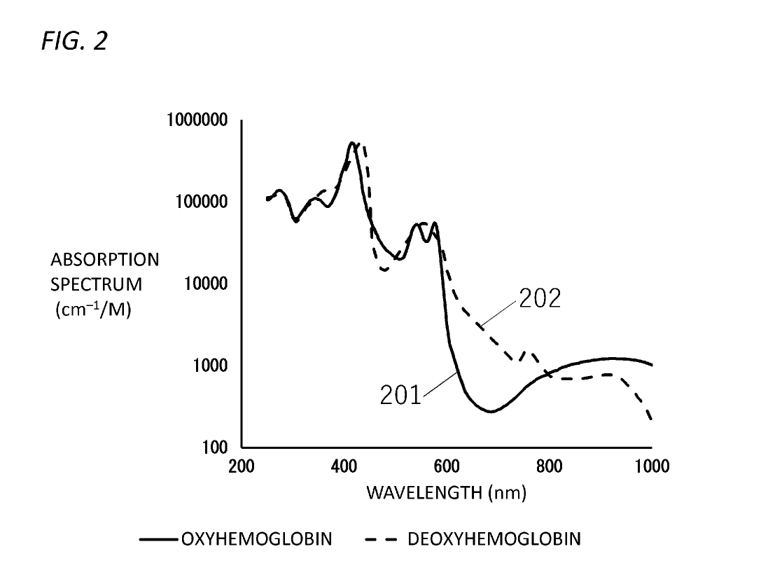

FIG. 2 is a graph that shows the absorption spectra of oxyhemoglobin and deoxyhemoglobin. The horizontal axis on the graph indicates wavelength (nm), while the vertical axis on the graph indicates the absorption spectrum (cm⁻¹/M). In this graph, the solid line indicated by reference sign 201 shows the absorption spectrum of oxyhemoglobin, while the dashed line indicated by reference sign 202 shows the absorption spectrum for deoxyhemoglobin. The occurrence ratio between oxyhemoglobin and deoxyhemoglobin within the object can be acquired by analysis of the acoustic waves obtained upon irradiation of the object with light at a wavelength where there is a difference between the absorption spectrum for oxyhemoglobin and the absorption spectrum for deoxyhemoglobin.

### First Embodiment

The photoacoustic apparatus according to this first embodiment is an apparatus that irradiates the object with light pulses and produces a blood vessel image (structural image) of the interior of the object upon receiving the photoacoustic waves generated within the object.

In addition, the photoacoustic apparatus according to the first embodiment has a diagnostic support function of performing the photoacoustic measurement on a body portion (breast, hands, feet, fingers, and so forth) that forms a left-and-right pair in the same individual and estimating the presence/absence of a disease (tumor) based on the acquired photoacoustic images.

<System Structure >

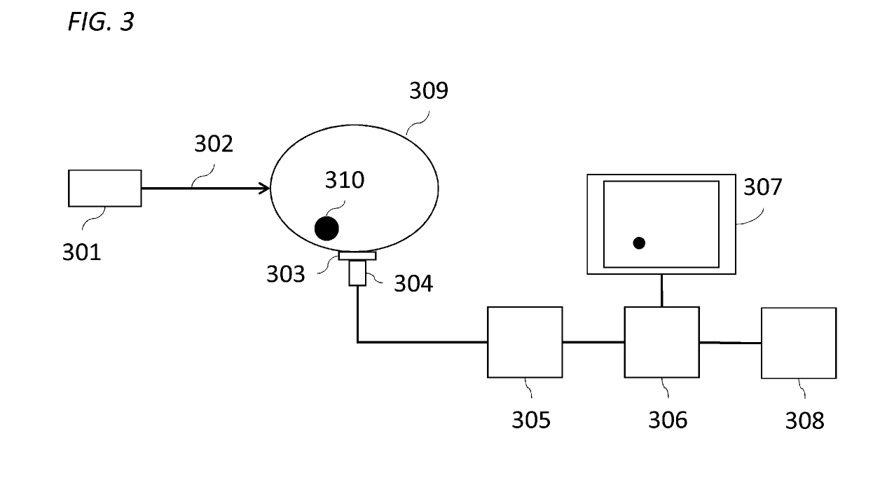

The structure of the photoacoustic apparatus according to the first embodiment is described in the following with reference to FIG. 3. The photoacoustic apparatus according to the first embodiment has the function of performing diagnostic support with respect to the breast.

The photoacoustic apparatus according to the first embodiment comprises for its constitution a light source 301, an optical system 302, an acoustic matching material 303, an acoustic wave probe 304, a signal processing section 305, a data processing section 306, and a display section 307. A data storage section 308 is connected to the data processing section 306 via a bus or a network. Reference sign 309 is the object, while reference sign 310 indicates a photoabsorber residing in the interior of the object.

The light source 301 is an apparatus that generates light pulses that are irradiated onto a subject. The light source is preferably a laser light source in order to obtain a high output, but a light-emitting diode or a flash lamp may also be used instead of a laser. When a laser is used as the light source, a variety of lasers can be used in this case, e.g., a solid-state laser, gas laser, dye laser, semiconductor laser, and so forth.

The wavelength of the light pulse preferably is a predetermined wavelength that is absorbed by a predetermined component among the components constituting the object and preferably is a wavelength at which the light propagates into the interior of the object. At least 700 nm and not more than 1200 nm is specifically preferred. Light in this region can reach to relatively deep sections of the body and can thus yield information on the deep sections.

In order to effectively generate photoacoustic waves, irradiation with the light must be performed for a sufficiently short time in conformity to the thermal characteristics of the object. When the object is a living body as in the present embodiment, the pulse width of the light pulses generated by the light source is advantageously not more than several tens of nanoseconds.

The timing, waveform, intensity, and so forth of the irradiated light is controlled by a control unit (not shown).

The optical system 302 is a member that transmits the light pulses generated from the light source 301. Using an optical component such as a lens or mirror, the light output from the light source 301 is guided to the object while being processed to a predetermined light distribution format and is irradiated. The light may also be transmitted using an optical waveguide such as optical fiber.

The optical system 302 may include, for example, optical elements such as lenses, mirrors, prisms, optical fiber, diffuser plates, shutters, filters, and so forth. Any optical component may be used in the optical system 302 as long as the light generated from the light source 301 can be irradiated in a desired form on the object 309. Viewed in terms of the safety for the organism and widening the diagnostic region, the light is preferably broadened in area to a certain degree rather than being focused with a lens.

The acoustic wave probe 304 is a unit that receives the acoustic waves incoming from the interior of the object 309 and converts these acoustic waves into an analog electrical signal. Acoustic wave probes are also referred to as probes, acoustic wave detection elements, acoustic wave detectors, acoustic wave receivers, and transducers. The acoustic waves generated from the body are ultrasound waves at from 100 kHz to 100 MHz, and due to this an element that can receive in this frequency band is used for the acoustic wave probe. Specifically, the following, for example, can be used: transducers that use a piezoelectric phenomenon, transducers that use light resonance, and transducers that use changes in capacitance.

An acoustic element that has a high sensitivity and a broad frequency band is preferably used for the acoustic element. Specific examples are piezoelectric elements that use, for example, lead zirconate titanate (PZT); polymeric piezoelectric film materials, e.g., of polyvinylidene fluoride (PVDF); capacitive micromachined ultrasound transducers (CMUT); and acoustic elements that use a Fabry-Perot interferometer. However, there are no limitations to just the acoustic elements indicated here, and any acoustic element that fulfills the functionality of a probe may be used.

An acoustic matching material 303, which is a member for matching the acoustic impedance, is disposed between the acoustic wave probe 304 and the object 309. For example, a gel, water, or an oil may be used for the acoustic matching material 303.

The signal processing section 305 is a unit that amplifies the acquired analog electrical signal and converts same to a digital signal. The signal processing section 305 may be constructed using an amplifier that amplifies the received signal, an A/D converter that converts the received analog signal to digital, memory, e.g., FIFO, which stores the received signal, and an arithmetic circuit, e.g., an FPGA chip. The signal processing section 305 may be constituted of a plurality of processors or arithmetic circuits.

The data processing section 306 is a unit (an image acquisition unit, index value acquisition unit, and acquisition unit in the present invention) that produces object information, i.e., the light absorption coefficient, oxygen saturation, and so forth in the interior of the object, based on the digitally converted photoacoustic signal. In specific terms, a three-dimensional initial acoustic pressure distribution for the interior of the object is produced based on the collected photoacoustic signal. For example, a universal back-projection (UBP) algorithm or a delay-and-sum algorithm can be used to produce the initial acoustic pressure distribution.

The data processing section 306 also produces a three-dimensional light intensity distribution for the interior of the object based on the dose of light irradiated on the object 309. The three-dimensional light intensity distribution can be acquired by solving the optical diffusion equation from the information pertaining to the two-dimensional light intensity distribution.

In addition to the preceding, the data processing section 306 has a functionality of producing a three-dimensional absorption coefficient distribution based on the initial acoustic pressure distribution and the light intensity distribution, and/or a functionality of producing an oxygen saturation distribution from the absorption coefficient distribution produced based on light at a plurality of wavelengths.

The data processing section 306 may be constituted of a computer having a CPU and RAM, nonvolatile memory, and a control port. Control is performed by the execution, by the CPU, of a program stored in the nonvolatile memory. The data processing section 306 may be realized by a general-purpose computer or a dedicated work station. In addition, the unit responsible for the computational functionality of the data processing section 306 may be constituted of a processor such as a CPU or GPU or an arithmetic circuit such as an FPGA chip. These units may be constituted of only a single processor or arithmetic circuit or may be constituted of a plurality of processors or arithmetic circuits.

The unit responsible for the storage function of the data processing section 306 may be a non-temporary storage medium, e.g., ROM, a magnetic disk, or flash memory, or may be a volatile medium such as RAM. The storage medium that stores the program is a non-temporary storage medium. These units may be constituted of one storage medium or may be constituted of a plurality of storage media. The unit responsible for the control function of the data processing section 306 is constituted of an arithmetic unit, e.g., a CPU.

The display section 307 is a unit that displays the information acquired by the data processing section and the processed information therefrom, and is typically a display device. The display section 307 may be provided with a plurality of display elements and may have a structure that enables parallel display.

The data storage section 308 is a storage unit constituted of, for example, a magnetic disk, and is connected to the data processing section through an internal bus or a network.

The object 309, while not constituting a part of the photoacoustic imaging apparatus, is described in the following. The photoacoustic imaging apparatus according to this embodiment can visualize, as an image, a condition within a body, e.g., a diseased part in a human or animal. Thus, an organism or body, and specifically, the breast, limbs, fingers, and so forth, may be contemplated for the object, but the breast is envisioned for this embodiment.

Blood vessels run through the interior of the breast and photoabsorbers 310 having a large light absorption coefficient, e.g., oxyhemoglobin and deoxyhemoglobin, are thus present, and as a consequence photoacoustic waves are produced due to irradiation with light. The hemoglobin distribution within the breast can be visualized by reconstructing these photoacoustic waves as an image. The blood vessel architecture can be visualized as an image because hemoglobin is present primarily in erythrocytes and erythrocytes are present within the blood vessels. The occurrence ratio between oxyhemoglobin and deoxyhemoglobin can also be visualized by irradiating light at a plurality of wavelengths.

<Summary of the Measurement >

A method for carrying out measurement on an organism, i.e., an object, using the photoacoustic apparatus according to this embodiment is described in the following.

First, light pulses generated from the light source 301 traverse the optical system 302 and are irradiated onto the object 309. When a portion of the energy of the light propagating through the interior of the object 309 is absorbed by the photoabsorber 310 in, e.g., the blood, acoustic waves are produced from the photoabsorber by thermal expansion. When cancer is present within the body, light is specifically absorbed at the new blood vessels of the cancer just as for the blood in the other normal regions and acoustic waves are thereby generated. The photoacoustic waves produced within the body are received by the acoustic wave probe 304.

The signal received by the acoustic wave probe 304 is converted by the signal processing section 305 and is subsequently analyzed by the data processing section 306. The results of the analysis provide volume data (photoacoustic data in the following) that expresses characteristic information on the interior of the body (for example, the initial acoustic pressure distribution or the absorption coefficient distribution), which, after conversion to a two-dimensional image, is output via the display section 307.

The photoacoustic data originating with the photoabsorber 310 and converted into data by the data processing section 306 is stored in the data storage section 308. Other information related to the object and measurement may be associated at this point. The data stored at the data storage section 308 includes, for example, the volume data acquired by the measurement, image data, information on the object, the parameters at the time of capture, and so forth. In the present embodiment, the photoacoustic measurement on the object is performed by the light source 301, the optical system 302, the acoustic wave probe 304, the signal processing section 305 and the data processing section 306, all of which constitute a photoacoustic measuring unit. The unit responsible for the image generating functionality among the functionalities of the data processing section 306 constitutes the photoacoustic measuring unit. As described below, the data processing section 306 also performs diagnostic support using images generated by the photoacoustic measuring unit. The unit responsible for the diagnostic support functionality using the images generated by the photoacoustic measuring unit among the functionalities of the data processing section 306 does not constitute the photoacoustic measuring unit.

The photoacoustic data stored at the data storage section 308 may be three-dimensional volume data or may be two-dimensional image data obtained from this data. For example, it may be data provided by the two-dimensionalization of the three-dimensional data using the maximum intensity projection method (MIP) or may be a two-dimensional image sliced out of the three-dimensional data at any plane or curved surface.

<Diagnosis Assist Method Using the Object Image>

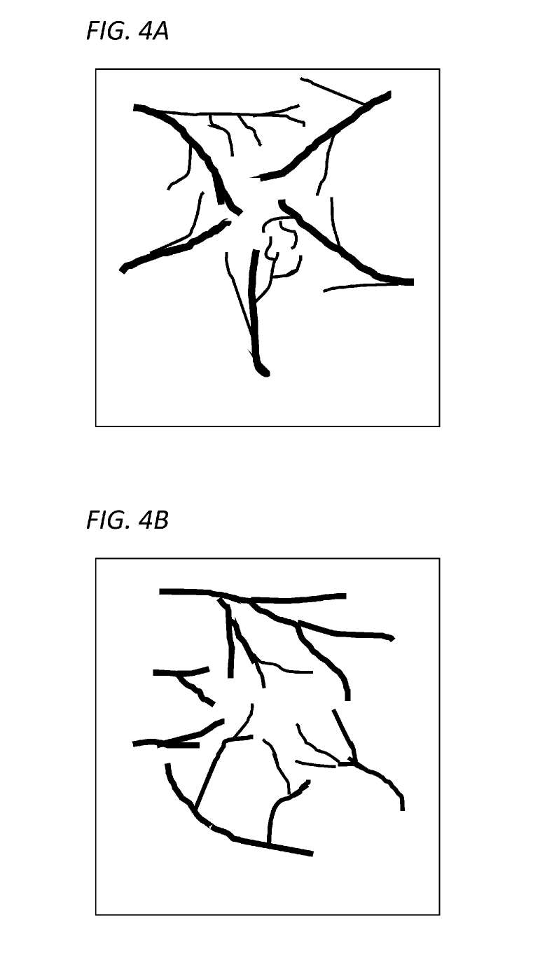

FIG. 4A and FIG. 4B are examples of object images obtained by carrying out photoacoustic measurement on the breast as the object. Specifically, this is an example in which light at a wavelength that supports absorption by hemoglobin is irradiated over the whole breast; volume data is generated based on the resulting acoustic waves; and visualization is effected by conversion into a two-dimensional image using the maximum intensity projection method. The images presented in FIG. 4A and FIG. 4B are images that correspond to the coronal plane with the viewpoint being the front direction.

FIG. 4A and FIG. 4B are, respectively, blood vessel architectures that have been imaged using the photoacoustic data acquired from the left and right breasts of the same individual.

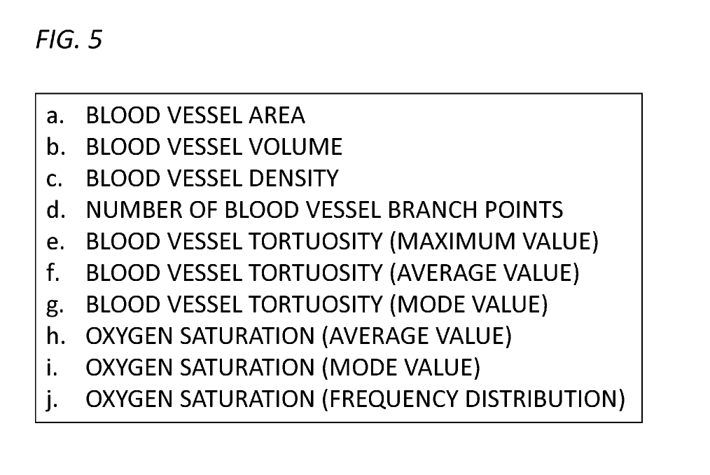

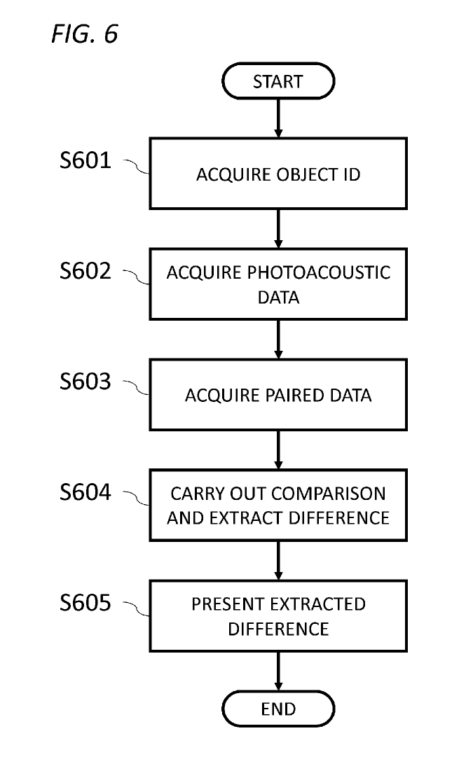

The photoacoustic apparatus according to this embodiment functions to provide—based on the photoacoustic images corresponding to the left and right breasts, respectively—information with respect to whether cancer is present in the breast. The flow for this functionality is described below with reference to FIG. 5 and FIG. 6.

The photoacoustic image in this Specification is not necessarily in a two-dimensional image format and may be three-dimensional volume data. Photoacoustic data and photoacoustic image are equivalent in the description that follows.

FIG. 5 is an example of the points of interest in a comparison of the image indicated in FIG. 4A with the image indicated in FIG. 4B.

It is generally thought that a disordered vascular network exists around a tumor (lesion) due to the action of the tumor itself. That is, the vascular structure around a tumor is more complex than at other portions. Thus, in a case where a vascular image can be visualized, as with a photoacoustic apparatus, it then becomes possible to estimate the potential that a tumor is present on the left or right by comparing a left object with a right object using, as an index value, a value that expresses a characteristic feature such as the amount or complexity of the blood vessels. That is, the data processing section 306—by comparing index values that express a characteristic feature within the regions of interest set for the respective plurality of images—can acquire information expressing the possibility of the presence of a lesion for at least one of the objects forming a pair.

For example, in a case where the acquired photoacoustic data is a two-dimensional image, the area of the blood vessels can be used as an index value. When the acquired photoacoustic data is three-dimensional volume data, the volume of the blood vessels can be used as an index value. Otherwise, the blood vessel density may be used. The area or volume of the blood vessels can be calculated by counting the number of pixels or voxels corresponding to blood vessels.

The number of blood vessel branch points and the blood vessel tortuosity may be used as complexity-related index values. These values can be quantified through image analysis.

The oxygen saturation of the periphery surrounding a tumor is hypothesized to be different from that of other portions. For example, the periphery surrounding a tumor is hypothesized to reside in a hypoxic condition relative to other portions. Comparison may therefore also be carried out using the oxygen saturation as an index value.

The oxygen saturation can be calculated from the ratio between oxyhemoglobin and deoxyhemoglobin. The oxygen saturation can be calculated with a photoacoustic apparatus by irradiating each object with light at a plurality of wavelengths that include light at a wavelength at which the absorptivities of oxyhemoglobin and deoxyhemoglobin are different.

Besides the oxygen saturation and the blood vessel area, volume, density, and complexity, any information can be used for the index value that expresses a characteristic feature within the region of interest insofar as this information presents differences depending on the possible presence of a lesion. The index value expressing a characteristic feature may not be blood vessel-related information, and any information can be used insofar as the information presents differences depending on the possible presence of a lesion.

FIG. 6 is a flow chart that shows the flow in the diagnostic support process. The process shown in FIG. 6 is started by a command from a reader (the user in the following) at the point when measurement of the object has been completed and the photoacoustic data has been collected.

The object ID is first acquired in the step S601 in order to designate the target object. The object ID may be input by the user or may be transmitted from, for example, the program that controls the apparatus.

The step S602 is a step of acquiring photoacoustic data for the target. The photoacoustic data acquired by the photoacoustic apparatus is stored, for example, in the data storage section 308 provided in the apparatus or in an externally connected storage device. In this step, data corresponding to the object designated in the step S601 is acquired from the stored data.

The photoacoustic data that forms a pair with the photoacoustic data retrieved in the step S602 is acquired in the step S603. For example, in a case where the data acquired in the step S602 is data representing the left breast of a particular patient, the data for the right breast of the same individual is acquired in the step S603. The paired data may be designated based on input from the user or may be automatically retrieved based on information associated with the photoacoustic data.

In the step S604, the photoacoustic data acquired in the step S602 and the photoacoustic data acquired in the step S603 are each analyzed and scored and the difference is extracted therefrom. As has been described in the preceding, the photoacoustic data to be compared may be three-dimensional volume data or may be two-dimensional images. A difference is extracted in this step by comparing a designated item, such as those provided as examples in FIG. 5. The data processing section 306 calculates information indicating differences of characteristic features between images, as the results of the comparison, such as a difference or a ratio of index values expressing characteristic features between paired images. The larger difference of the index values expressing characteristic features between the images indicates that the difference of characteristic features between the images is larger. The ratio of the index values more away from 1 indicates that the difference of characteristic features between the images is larger.

The comparison procedure carried out in the step S604 is described here in greater detail with reference to FIG. 4A and FIG. 4B. As has been described above, FIG. 4A and FIG. 4B are, respectively, visualizations of the vascular architecture, as two-dimensional images in the coronal plane, using the photoacoustic data acquired from the left and right breasts of the same individual.

Since these images are images of the left and right breasts captured at different times, standardization of the data is preferably carried out in a case where a comparison is to be performed.

Data standardization may be performed here by setting regions of interest whereby the left and right data become approximately equivalent. For example, in a case where a comparison is to be carried out using two-dimensional images such as those shown in FIG. 4A and FIG. 4B, a method may be used in which regions of interest are set whereby the contained areas are the same.

Examples of methods for setting the region of interest are as follows.
(1) a method in which the regions of interest are manually set for both of the two images
(2) a method in which a reference point is set in one image and a region of interest of approximately the same area containing this reference point is set in both images
(3) a method in which regions of interest of approximately the same size are automatically set in both images

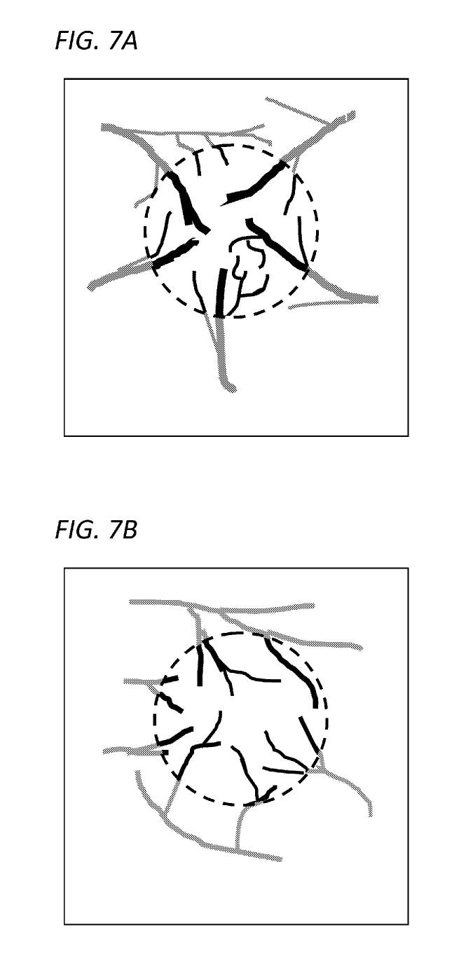

As an example, FIG. 7A and FIG. 7B give an example in which, for the images provided in FIG. 4A and FIG. 4B, regions of interest have been set in the form of circular regions having the same radius for which the center is the nipple. The position of the nipple that is set as the center may be designated by the user or, in a case where the apparatus has information on the position of the nipple, may be automatically set based on this information. In addition, the coordinate information for the position of the nipple may be stored as metadata in, for example, a header for the photoacoustic data.

The nipple is used as the reference point because the nipple is a characteristic point present in common in the center of the breast and it thus enables the performance of a meaningful comparison of the surrounding blood vessels.

A circular region is used for the region of interest in FIG. 7A and FIG. 7B, but a non-circular region may be used for the region of interest in conformity with the configuration of the acoustic wave probe provided with the photoacoustic apparatus.

Moreover, in this example, the region of interest has been set so the areas for the left and right objects are equal; however, in a case where the photoacoustic data for the target is three-dimensional data, the region of interest may be set so the volumes for the left and right objects are equal.

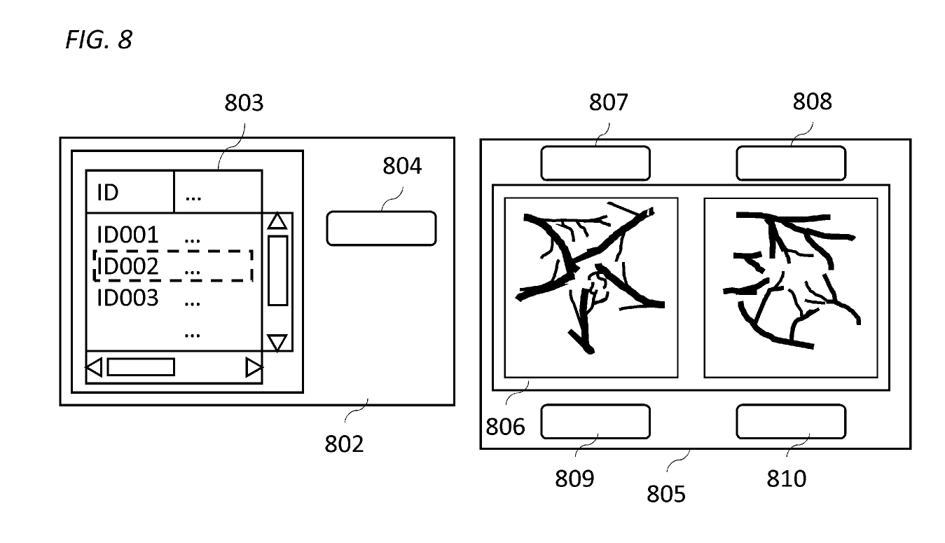

FIG. 8 is an example of an operations screen for setting the region of interest.

An identification number is set for each object in the photoacoustic data stored in the data storage section 308. In a list display screen 802, the photoacoustic data stored in the data storage section 308 is displayed as a list by identification number at a list display section 803.

On the list display section 803, the identification number for the data to be displayed on the screen is selected with, for example, a mouse, and the corresponding photoacoustic data can then be displayed on the display screen 805 by pressing the call command button 804. A button-driven operation need not be used, and an operation on the list display section 803, such as a mouse double click, may be used, or a menu-driven operation may be used.

When the imaged photoacoustic data is stored in the data storage section 308 as data for a left-and-right pair, the images forming the pair are automatically acquired and are displayed simultaneously at an image display area 806.

In this example, using the reference point as the center, regions of interest are set for one left-and-right pair of image data displayed on the display screen 805. The reference point may be set by the user, or may be automatically extracted in a case where data pertaining to the reference point is appended to the photoacoustic data. In addition, the reference point may be automatically set by detection of the reference point.

For example, information pertaining to the reference point may be read out from the photoacoustic data and a button (reference sign 807, 808) may be established in order to set the region of interest. In addition, a button (reference sign 809, 810) for detection of the reference point through image analysis may be established.

Information indicating that the photoacoustic data displayed at the display area 806 is data that forms a left-and-right pair for the same object is stored, for example, in control data for the photoacoustic apparatus, a header section of the photoacoustic data, or a header section for data that is imaged photoacoustic data. When one of the data is selected by the user, the paired data can be automatically selected using these data, but the paired data may also be explicitly selected by the user.

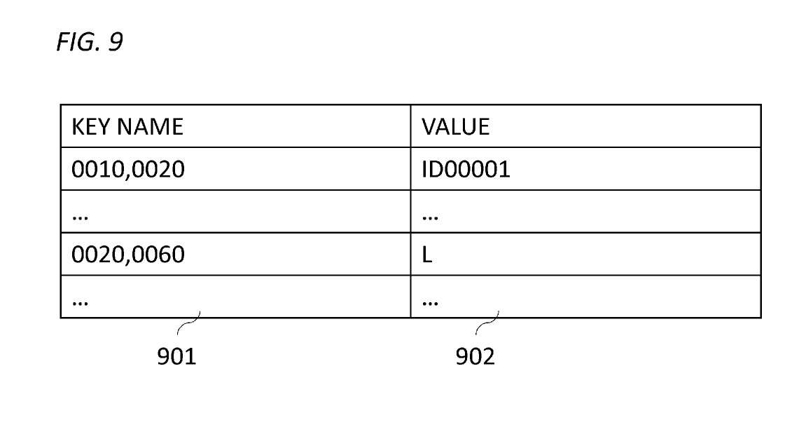

In the first embodiment, information indicating that either the left or right has been captured is stored in a header section for the photoacoustic data. The DICOM Standard is an example of an image format that can store appended information such as this. FIG. 9 is an example of header information according to an image format that conforms to the DICOM Standard.

In the data structure shown in FIG. 9, reference sign 901 is the DICOM tag and reference sign 902 is the value corresponding to the DICOM tag. In this example, by assigning reference numbers “0020 (indicating examination-related information)” and “0060 (indicating left/right information)” to the DICOM tag and storing data that sets “L” as its value, the meaning is indicated that the image having this header section is an image of the left chest.

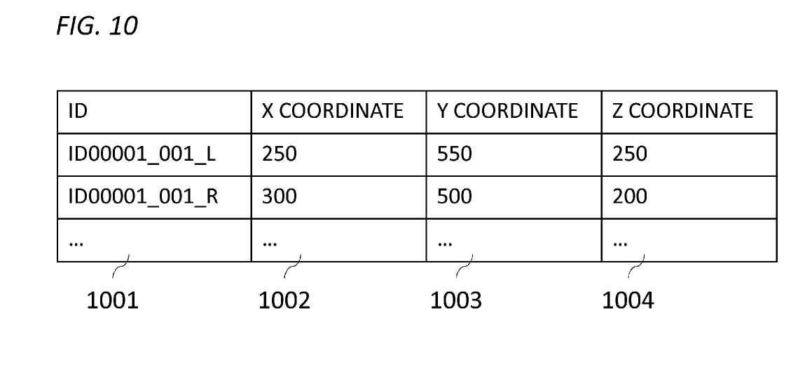

FIG. 10 is an example of a table for the case of storage in the data storage section of information representing the reference point.

In FIG. 10, reference sign 1001 is an ID that uniquely identifies the photoacoustic data of the target. Reference signs 1002, 1003, and 1004 are coordinates for the reference point in the photoacoustic data. With regard to the structure of this table, it may be stored in the data storage section as an independent table that holds only information on the reference point, or it may be a part of a table that holds other information on the photoacoustic data.

The example given in FIG. 10 is an example in which the position of the reference point is stored as coordinates in three-dimensional space, but the position of the reference point may be two-dimensional data in a case where the photoacoustic data is handled as a two-dimensional image.

In the step S605, information is presented to the user based on a difference obtained by comparing the paired photoacoustic data. In this step, for example, the difference for each item given in FIG. 5 may be presented, or the results of a global evaluation may be presented. The user can render a diagnosis making reference to the information presented in the step S605.

The results of the comparison may be reported numerically or graphically. For example, each of the acquired index values (absolute values) may be reported side-by-side, or relative values (e.g., percentages) may be reported. The possibility of the presence of a tumor may be estimated based on the results of the comparison, and related information may also be output. For example, in a case where a threshold value has been pre-stored and the difference in the index values exceeds the threshold value (a difference equal to or greater than a predetermined value is observed), an indication that a tumor may be present may be output. In this case, the probability of the presence of the tumor may also be calculated and output at the same time. The threshold value may be pre-set or may be input by the user.

In addition, in a case where the photoacoustic data and data on the presence/absence of a tumor have been collected, the threshold value may be determined by carrying out machine learning using these as teaching data. Or, in a case where the index values for the left and right are input, a discriminator that outputs the tumor occurrence potential may be generated and used.

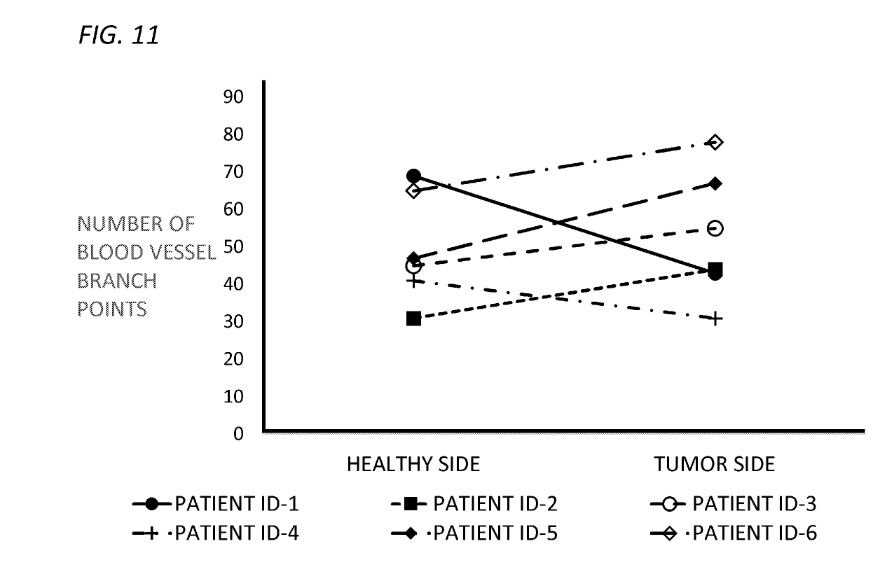

FIG. 11 contains a graph of the results provided in a case where analysis was performed targeted on the regions of interest set in images corresponding to the right and left breasts. In this example, the blood vessels were visualized with a photoacoustic apparatus in the right and left breasts of breast cancer patients, and the number of blood vessel branch points, taken to be an index value expressing a characteristic feature within the region of interest, was compared.

Numerous newly produced blood vessels are seen in a case where a tumor is present. That is, in a case where there is more blood vessel branching in one breast than in the other, the potential presence of cancer can be estimated for the breast on that side.

In a case where, as shown in FIG. 11, there is a substantial difference, between the diseased side and the non-diseased side, in the distribution of the index values obtained from the regions of interest, this index value may then function as an index for discriminating the presence/absence of disease. In this case, by comparing the left and right index values for a patient who has not received a diagnosis with regard to the presence/absence of disease, supplementary information can be obtained at the time of diagnosis.

With a photoacoustic apparatus, there is a limit on the depth (the depth limit) to which the light irradiated onto the object may reach, and thus a photoabsorber present at a location beyond the depth limit is not visualized. However, in a case where a disease is present that, accompanied by angiogenesis, exercises a substantial influence on vascular architecture, such as a tumor or a site of inflammation, characteristic features of the blood vessels present at less than the depth limit present slight variations even though the disease site resides at a position deeper than the depth limit. These variations can be detected with the photoacoustic apparatus according to the present embodiment notwithstanding the difficulty of discriminating such variations with the naked eye. That is, even though the tumor itself is not visualized, supplementary information for evaluating the presence/absence of a tumor can be provided.

### Second Embodiment

The second embodiment is an embodiment in which diagnostic support is performed with regard to the presence/absence of lymph node involvement in breast cancer. The apparatus structure and so forth of the photoacoustic apparatus according to this second embodiment conforms to the first embodiment.

In this second embodiment, photoacoustic data is acquired using left and right sentinel lymph nodes as the target. In the first embodiment, the region of interest is set in the step S605 using the nipple as the reference (characteristic point); however, in the present embodiment the region of interest is set using a sentinel lymph node as the reference. In addition, the index values shown in FIG. 5 are acquired and a comparison is performed between regions of interest for which the references (characteristic points) are the left and right sentinel lymph nodes. When, as a result, a significant difference is observed between the left and right index values, a finding of suspicion of breast cancer metastasis to the sentinel lymph nodes can then be made.

### Third Embodiment

The third embodiment is an embodiment in which the results determined by the apparatus in the first and second embodiments are externally output.

In this third embodiment, the performance of the step S605 is followed by the performance by the data processing section 306 of a step of producing the evaluation results and externally outputting same.

Specifically, for example, data containing the information as indicated below is produced and is written to the data storage section 308 associated with the apparatus or is transmitted to an external management unit (not shown).
object ID
portion identifier (hand, foot, breast, and so forth)
left/right identifier
right index value/left index value (absolute values)
value expressing difference between left and right (relative value)
evaluation results (presence/absence of tumor, possible presence of tumor, and so forth)

The output data may be produced, for example, by a controllable format by a PACS (health information and management system). In addition, in a case where the photoacoustic data is in the DICOM format, this information may be written to a header. This information can be written since there are private fields in the DICOM header. The photoacoustic data in which the information has been written may be rewritten to the data storage section 308, or, in those instances in which the photoacoustic data was acquired from an external device, may be transmitted to the external device.

(Examples of Modifications)

The description in each embodiment is an example with regard to describing the present invention, and the present invention may be executed using appropriate alterations and combinations within a range that does not depart from the essential features of the invention.

For example, the present invention may also be executed as a photoacoustic apparatus that includes at least a portion of the units described in the preceding. The present invention may also be executed as an information processing apparatus that omits the photoacoustic measuring unit for performing photoacoustic measurements on an object. That is, it may be an apparatus that receives and processes photoacoustic data from an external source.

The present invention may also be executed as an information processing method that includes at least a portion of the processes described in the preceding. The processes and units described in the preceding may be freely combined and implemented as long as a technical conflict is not produced.

The breast was used as the object in the description of the embodiments, but, for example, the hands, feet, fingers, and so forth may be used as the object. Diagnostic support using difference information from the photoacoustic data is made possible by using a body portion that forms a pair as the target.

Data acquired using a photoacoustic apparatus is used in the description of the embodiments, but application to other modalities is also possible. For example, application to angiographic images, nuclear magnetic resonance images, tomographic images, and so forth is also possible.

The description of the embodiments has concerned the example of a comparison of the left and right breasts, but when information acquired in the past exists, additional comparisons may be made using that information. For example, the data storage section 308 may store information on index values previously acquired from the left and right breasts, respectively. Comparisons may be run using the stored past index values. The results of the comparisons may be displayed numerically or may be displayed visually as shown in FIG. 11.

The embodiments described above concern an example in which information expressing the possible presence of a lesion is generated based on image data for objects that correspond to portions that form a pair with each other, but other information pertaining to the condition of the object may be generated based on this image data. For example, the data processing section—by comparing characteristic quantities contained in the image data of objects that correspond to portions that form a pair with each other—may generate information indicating the degree of malignancy of the tumor as information indicating the condition of the object. Information indicating the possibility of the presence of a lesion in the object may also be included in the information indicating the condition of the object. The information indicating the condition of the object may also be handled by the apparatus according to the present invention in the same manner as the information indicating the possible presence of a lesion.

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2017-190719, filed on Sep. 29, 2017, which is hereby incorporated by reference herein in its entirety.

What is claimed is:

1. An information processing apparatus, comprising:
an image acquisition unit that acquires first image data for a first object and second image data for a second object that corresponds to a portion that forms a pair with the first object;
a setting unit that sets a region of interest for the first image data and a region of interest for the second image data;
an index value acquisition unit that acquires an index value that represents a characteristic feature within the region of interest for the first image data and an index value that represents a characteristic feature within the region of interest for the second image data; and
an acquisition unit that, based on the results of a comparison of the index values, acquires information indicating the possible presence of a lesion in at least one of the first object and the second object.

2. The information processing apparatus according to claim 1, wherein the acquisition unit acquires the information indicating the possible presence of a lesion in a case where the difference between the index values to be compared is equal to or greater than a predetermined value.

3. The information processing apparatus according to claim 1, wherein the index value is a numerical value that indicates the amount or complexity of blood vessels present in the region of interest.

4. The information processing apparatus according to claim 1, wherein the first object and the second object respectively correspond to body portions that form a left and right pair.

5. The information processing apparatus according to claim 4, wherein the setting unit sets the region of interest based on a characteristic point that is present in common in both left and right of the body portions.

6. The information processing apparatus according to claim 5, wherein
the first object and the second object respectively correspond to a left breast and a right breast, and
the characteristic point is a point corresponding to a nipple or a sentinel lymph node.

7. The information processing apparatus according to claim 5, wherein
the first image data and the second image data are three-dimensional volume data, and
using the characteristic point as the center, the setting unit sets a range over which volumes of the first object and the second object are approximately the same, as the region of interest.

8. The information processing apparatus according to claim 5, wherein
the first image data and the second image data are two-dimensional images, and
using the characteristic point as the center, the setting unit sets a range over which areas of the first object and the second object are approximately the same, as the region of interest.

9. The information processing apparatus according to claim 4, wherein the first image data and the second image data are images conforming to the DICOM Standard, and information pertaining to the body portion is contained in a DICOM header.

10. The information processing apparatus according to claim 1, wherein the acquisition unit outputs the information indicating the possible presence of a lesion as data that is appended to at least one of the first image data and the second image data.

11. The information processing apparatus according to claim 10, wherein
the first image data and the second image data are images conforming to the DICOM Standard, and
the acquisition unit outputs the information indicating the possible presence of a lesion, to a DICOM header of at least one of the first image data and the second image data.

12. The information processing apparatus according to claim 1, wherein the first image data and the second image data are data generated by a photoacoustic measuring unit.

13. The information processing apparatus according to claim 1, wherein the acquisition unit acquires a probability that a lesion is present as the information indicating the possible presence of a lesion.

14. A photoacoustic apparatus, comprising:
a photoacoustic measuring unit that generates the first image data and the second image data based on acoustic waves generated by objects by irradiating the objects with light; and
the information processing apparatus according to claim 1.

15. An information processing method, comprising the steps of:
acquiring first image data for a first object and second image data for a second object that corresponds to a portion that forms a pair with the first object;
setting a region of interest for the first image data and a region of interest for the second image data;
acquiring an index value that represents a characteristic feature within the region of interest set for the first image data and an index value that represents a characteristic feature within the region of interest set for the second image data; and
acquiring, based on the results of a comparison of the index values, information indicating the possible presence of a lesion in at least one of the first object and the second object.

16. A non-transitory computer readable medium that stores a program, wherein the program causes a computer to execute:
acquiring first image data for a first object and second image data for a second object that corresponds to a portion that forms a pair with the first object;
setting a region of interest for the first image data and a region of interest for the second image data;
acquiring an index value that represents a characteristic feature within the region of interest set for the first image data and an index value that represents a characteristic feature within the region of interest set for the second image data; and
acquiring, based on the results of a comparison of the index values, information indicating the possible presence of a lesion in at least one of the first object and the second object.
